(19) United States
(12) Patent Application Publication
(10) Pub. No.: US 2010/0256430 A1
(43) Pub. Date: Oct. 7, 2010

(54) SYSTEM AND PROCESS FOR THE TREATMENT OF RAW MATERIAL

(75) Inventor: Christopher Francis Bathurst, Christchurch (NZ)

Correspondence Address: MORRISON & FOERSTER LLP, 1650 TYSONS BOULEVARD, SUITE 400, MCLEAN, VA, 22102, US

(73) Assignee: SOLRAY ENERGY LIMITED, INVERCARGILL (NZ)

(21) Appl. No.: 12/743,282

(22) PCT Filed: Nov. 17, 2008
(86) PCT No.: PCT/NZ08/00309

§ 371 Date: May 17, 2010

(30) Foreign Application Priority Data
Nov. 15, 2007 (NZ) 563472

(51) Int. Cl. C07C 1/00; A62D 3/37; F28D 21/00

(52) U.S. Cl. 585/242; 588/316; 588/319; 585/240; 422/208

(57) ABSTRACT

The invention provides a system and process for treating a raw material to reduce the level of a contaminant in the raw material or to form a raw product that can be used to produce a fuel.

The system and process include means for pressurising a pumpable feedstock comprising the raw material to a pressure of between 100 and 350 bar, preferably using indirect pressurising means; heating the feedstock to a temperature of between 250° C. and 400° C. within a processing vessel to form a pressurised raw product stream; cooling the raw product stream within the processing vessel to an ambient or near ambient temperature; then depressurising the raw product stream before discharging the raw product from the system.

### CROSS-REFERENCE TO RELATED APPLICATIONS

This application is a national stage application under 35 USC 371 of International Application No. PCT/NZ2008/000309, filed Nov. 17, 2008, which claims priority from New Zealand Patent Application No. 563472, filed Nov. 15, 2007, the contents of which prior applications are incorporated herein by reference.

### FIELD OF THE INVENTION

The invention relates to a system and process for removing contaminants from raw materials, such as organic and waste feedstock, and for treating raw materials to produce material from which a fuel can be produced.

### BACKGROUND OF THE INVENTION

There are an increasing number of contaminated materials being produced by industrial and commercial processes. This, combined with tighter legislative controls being imposed on the types and concentrations of contaminants allowed to be disposed of, has led to an increase in the variety of waste treatment processes now available.

Even so, there is an increasing quantity of waste materials that continue to be produced with low levels of contamination, where the bulk of the contaminant has been recovered and recycled. The remaining contaminant is typically either uneconomical to recover or is bound to the waste in some way. One example of this type of waste material is drycleaning waste, which contains a small quantity of perchloroethylene that is not economical to recycle, and cannot be legally disposed of in a landfill.

It is known that some contaminants can be removed from contaminated materials by oxidation. However, this often requires a strong oxidant, which must be carefully chosen to avoid creating additional contaminants within the waste or treated material. For example, if a chlorinated oxidant is used, there is a risk of stable chlorinated by-products being generated. In addition, many strong oxidants need to be stored and transported carefully, which adds to the cost of handling these oxidants and presents safety hazards. Because of these drawbacks, some oxidants are now generated on-site, and often in situ. One such oxidant is ozone. However, ozone carries with it additional problems because the ozone thus produced is a poisonous and highly reactive gas and, therefore, it cannot be allowed to escape from the process. To remove any residual ozone, various thermal and adsorption methods are used, but all of these add to the cost and complexity of the decontamination process.

Often, the treatment of waste material reduces the volume of contaminated material by concentrating the waste material, such as when the waste material is concentrated by drying. This concentrated waste may reduce the volume to be disposed of, but it can create additional problems if the contaminants in the waste material are to be removed.

For example, it can be difficult to remove contaminants from process materials at high temperatures (above about 250° C.) and at high pressures and the processing equipment, especially valves, may be expensive or may not be suitable to handle both high temperatures and high pressures. This drawback can prevent high pressure/temperature processes from being industrially applied or developed. For example, bench scale processes may not be scalable due to the availability of suitable equipment and fittings that can operate at high temperatures and high pressures. In addition, many bench scale operations do not scale well to larger commercially viable sizes.

Therefore, there is a need to provide a system and method for removing contaminants from waste material in an effective and efficient manner.

Another problem in today's society is the sustainability and environmental suitability of the fuel that we use. Because of the increasing environmental concerns associated with the combustion of hydrocarbons, and the variable cost of oil, the suitability of alternative fuels is being investigated and is gaining acceptance in some areas.

It is known to produce raw product material that can be used in the production of alternative fuels by subjecting particular raw materials to high temperatures and pressures.

However, known systems and processes for producing raw product materials are slow and inefficient and are also unable to accurately pressurise and depressurise the raw material to the required pressure. This is because the types of valves currently available are designed to seal well against high pressures, but only at low temperatures. These valves are unreliable for sealing at high temperatures. This is because any sealing and gland materials that have sufficient flexibility to form a good seal do not survive at high temperatures.

Therefore, there is a need to provide a system or process for producing a raw product material for an alternative fuel, where the system or process can accurately pressurise and depressurise the raw material using known valves and without requiring expensive specialist equipment to be developed.

### OBJECT OF THE INVENTION

It is an object of the invention to provide a high pressure and high temperature process and/or system for treating waste material and/or organic material that goes at least some way toward overcoming one or more of the deficiencies described above; or to at least provide a useful choice over present processes and systems.

### SUMMARY OF THE INVENTION

The invention provides a high pressure hydrothermal processing system and process for treating organic and/or waste materials. The processing system includes a pressurising section, a processing section and an output section. In operation, the pressurising section pressurises a charge of a feedstock to between 100 bar and 350 bar, the processing section heats and processes the pressurised feedstock at between 250° C. and 400° C. then cools a resultant product stream, and the output section depressurises the product stream before discharging the product.

The feedstock is a pumpable form of the raw material.

In one aspect, the invention provides a system for processing a raw material in the form of a pumpable feedstock, the system comprising:
a) a pressurising section comprising pressurising means for pressurising the feedstock to a pressure in the range of 100 to 400 bar;
b) a processing section comprising processing means for heating the feedstock to a temperature in the range of 250° to 400° C. by heating means to form a raw product stream and in which the processing means is adapted to allow the raw product stream to be cooled to an ambient or near ambient temperature before exiting the processing section; and
c) an output section in which the raw product stream is depressurised by depressurising means.

Preferably, the pressurising section comprises a first pump and the pressurising means comprises a second pump, the first pump being configured to draw feedstock from a feed tank and provide an initial low pressurisation and the second pump being configured to pressurise the feedstock.

More preferably, the pressurising section further comprises an additive pump for adding one or more additives to the feedstock to form a feedstock and additive mixture before the mixture is pressurised by the second pump.

Preferably, the processing means comprises at least one processing vessel comprising an inlet to a first stage connected with a reaction zone that is heated by the heating means, the reaction zone being connected to a second stage leading to an outlet through which reacted feedstock, in the form of a raw product stream, is able to exit the pressure vessel, wherein the inlet, first stage, reaction zone, second stage, and outlet form a fluid pathway along which the feedstock, and then the raw product stream, can pass through the pressure vessel.

More preferably, the processing vessel comprises a pressure vessel comprising a first end and a second end, wherein an inlet is positioned at or near the first end of the pressure vessel, the inlet being connected to a first tube positioned concentrically within a second tube, wherein the second tube forms the casing of the pressure vessel; and wherein the first tube comprises a distal end that terminates before the second end of the pressure vessel to form a reaction zone between the distal end of the first tube and the second end of the pressure vessel.

In a preferred form, the processing means comprises a counter-flow heat exchanger.

Preferably, the output section comprises a high pressure gas separator for separating out gases from the raw product stream, and further comprises a purge valve through which the gases can exit the system.

Preferably, the pressurising section comprises a second pump that is adapted to pressurise the feedstock using indirect pressurisation, the second pump comprising a housing in which a floating ram is located, wherein one side of the ram is adapted to be in contact with a fluid output of a separate pumping system, and the other side of the ram is adapted to be in contact with the feedstock within the housing, such that pressurising the fluid within the housing causes the ram 18 to press against the feedstock to pressurise the feedstock

Preferably, the depressurising means comprises a third pump adapted to depressurise the product stream using indirect depressurisation, the third pump comprising a housing in which a floating ram is located, wherein one side of the ram is adapted to be in contact with a fluid output of a separate pumping system, and the other side of the ram is adapted to be in contact with the raw product stream, and wherein the housing includes a release valve through which the fluid within the housing can exit the housing to allow the raw product stream to be depressurised.

In another aspect, the invention provides a continuous process for treating a raw material, the process comprising the following steps, in order:
a) prepare a pumpable feedstock or slurry from a raw material;
b) pressurise a charge of the feedstock to between 100 bar and 350 bar;
c) transfer the pressurised feedstock to a processing means;
d) raise the temperature of the pressurised feedstock to between 250° C. and 400° C. within the processing means to form a pressurised raw product stream;
e) cool the raw product stream within the processing means to ambient or near ambient temperatures; and
f) depressurise the raw product stream before discharging the raw product stream from the system.

Preferably, the raw product stream is cooled in the processing means to a temperature below 80° C.

More preferably, the raw product stream is cooled in the processing means to a temperature below 50° C.

One or more additives may be introduced to the pumpable feedstock before the feedstock and additive mixture is pressurised.

Each additive may be selected from the group consisting of: acids, bases, oxidising agents, and reducing agents.

Alternatively, each additive may be selected from the group consisting of: carbonates, hydroxides, bicarbonates, and similar bases.

In a preferred form, an additive 14a of sodium carbonate or calcium carbonate is added to the feedstock to form a feedstock and additive mixture containing at least 5% additive.

Preferably, the feedstock is pressurised by indirect pressurisation.

Preferably, the raw product stream that exits the processing means is then passed through a gas separator in which gas is purged from the raw product stream.

In a preferred form, the processing time between charges of feedstock is a minimum of 20 seconds.

In another preferred form, the feedstock comprises algae to which is added an additive of calcium carbonate to form a feedstock and additive mixture, which is then pressurised to 150 bar and heated to a temperature of approximately 320° C.

### BRIEF DESCRIPTION OF THE DRAWINGS

Preferred forms of the invention will now be described by way of example only and with reference to the accompanying drawings, in which:

FIG. 1 is a diagrammatic view of a system for high pressure, high temperature processing of waste and/or organic material according to one embodiment of the invention;

FIG. 2 is a diagrammatic view of the system for high temperature, high pressure processing of waste and/or organic material that includes preparation and separation stages according to one embodiment of the invention; and

FIG. 3 is a flowchart of a process using a high temperature hydrothermal system according to one embodiment of the invention.

### DETAILED DESCRIPTION OF PREFERRED FORMS OF THE INVENTION

The invention relates to a system and method for the high pressure, high temperature treatment of organic and waste feedstock such as dry cleaning sludge, biosolids sludge, de-lignitised timber and algae to: produce useful hydrocarbons, or material from which hydrocarbons can be removed, for the production of alternative fuels; and/or to remove contaminants from contaminated raw material.

For clarity, the term “high pressure”, when used in this specification and claims, refers to pressures above 100 bar.

The term “charge”, when used in this specification and claims, refers to a preset volume of feedstock.

The term “indirect pressurisation”, when used in this specification and claims, refers to pressurisation of a substance, such as a feedstock, by a pressurising means where the substance is pressurised due to the movement of an actuator, or ram, which moves consequent to the pressurisation of other fluid within the pressurising means.

Referring to the drawings, a high pressure hydrothermal processing system 1, including a pressurising section 2, a processing section 3 and an output section 4, is shown. The pressurising section 2 pressurises a feedstock 7 to be processed; the processing section 3 heats and processes the pressurised feedstock 7, then cools a resultant raw product stream; and the output section 4 depressurises and outputs the product stream 8.

It should be noted that the system and process can be used to remove contaminants from a feedstock or the system and process can be used to produce a product, such as a product containing hydrocarbons or crude oil, that may be suitable for use as a fuel. Thus, the product stream 8 may contain a desirable product produced in the processing section 3 or the product stream may be a material that is free from, or has a lower level of, contaminants that were removed by the process of the invention.

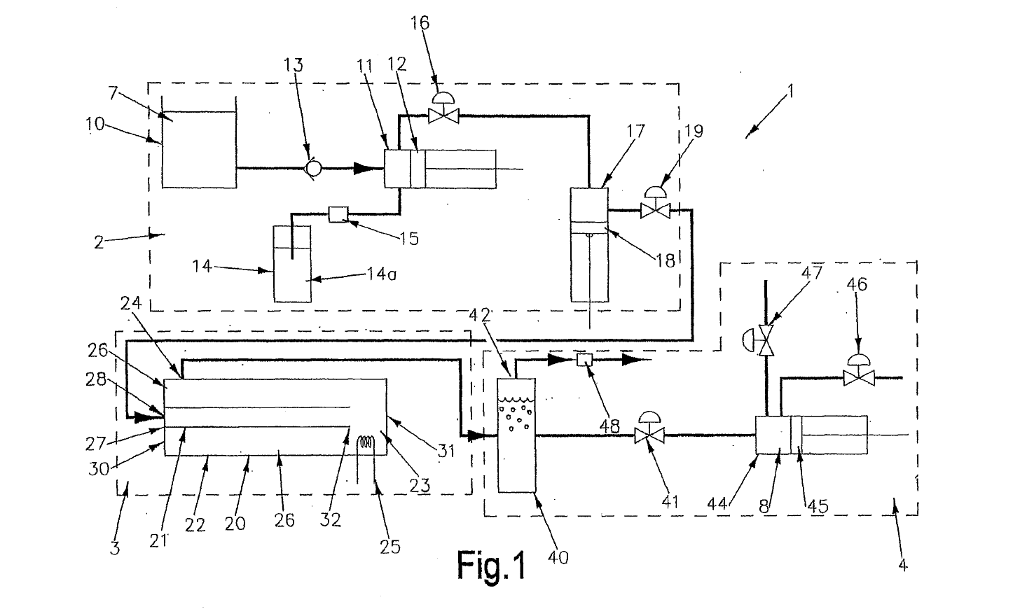

The system of the invention will be described first by reference to a preferred embodiment, as shown in FIG. 1. In this embodiment, the system includes a pressurising section 2 in which feedstock enters the system and is pressurised before being processed by the processing section 3. The pressurising section 2 includes a feed tank 10 connected to a first pump 11 via a conduit on which is located a non-return valve.

In the embodiment of FIG. 1, the first pump includes a first ram 12 that moves up and down within a cylinder and that is driven by any suitable means. However, if alternative forms of pump are used, the ram may be replaced with other suitable pumping means as would be apparent to a person skilled in the art.

The first pump 11 is configured to draw feedstock 7 from the feed tank 10 and provide an initial low pressurisation. For example, the feedstock can be drawn from the feed tank by moving the ram to create a vacuum. This causes the feedstock to move from the feed tank 10 to the first pump 11 via the conduit and non-return valve. The non-return valve prevents the feedstock from moving back toward the feed tank 10.

The pressurising section 2 also optionally contains an additive tank 14, adapted to contain an additive 14a. The additive tank is connected with an additive pump 15 that pumps one or more additives to the first pump 11 via a conduit that connects the additive tank to the first pump. This creates a feedstock and additive mixture in the first pump 11.

A first valve 16 is positioned on a conduit connected with the first pump 11 and with pressurising means, in the form of a second pump 17. The first valve can be closed to allow the first pump 11 to mix the feedstock with the additive within the pump 11, and the valve can be opened to allow the feedstock/mixture to be pumped from the first pump 11 to the second pump 17 via the conduit.

The second pump 17 is a high pressure pump that includes a pump housing in the form of a cylinder within which a second floating piston or ram 18 is located. The second ram 18 is able to slide back and forth along the cylinder in the usual manner. If alternative forms of pump are used, the ram may be replaced with other pumping means as would be apparent to a person skilled in the art.

The second pump 17 is configured to pressurise the feedstock 7. In particular, the pump 17 is configured so that one side of the ram is adapted to be in contact with a pure fluid, such as pure water, which has been pressurised independently by a conventional separate pumping system connected with the second pump. The other side of the ram is adapted to be in contact with the feedstock or feedstock and additive mixture, as the case may be, held within the cylinder or pump housing.

The system works by pumping the feedstock into the cylinder of the second pump 17 by opening the first valve 16 and actuating the first pump 11. As the feedstock/mixture enters the second pump, the second ram 18 is caused to move along the cylinder and push the pure fluid out from the other end of the cylinder and into a reservoir, not shown, via an open release valve.

The second pump 17 is also connected with a second valve 19. After the feedstock is pumped into the second pump 17 by the first pump 11, the first and second valves 16 and 19 are closed.

The pure fluid remaining in the second pump is then pressurised by the separate pump. This causes the ram 18 to transmit the pressure of the fluid by pushing against the feedstock/mixture, thereby pressurising the feedstock/mixture in an indirect way.

The second valve 19 can then be opened to allow the pressurised feedstock/mixture to be moved from the second pump 17 to the processing section 3.

The first and second valves 16, 19; first and second pumps 11, 17; and first and second rams 12, 18, all form part of the pressurising section 2.

Although the indirect pressurisation has been described in relation to a pump having a cylinder with a floating ram therein, other forms of pump may be used instead, as would be appreciated by a person skilled in the art.

Optionally, the system may be adapted to allow the feedstock to be moderately pre-heated in the pressurising section by including heating means along a section of the conduit, or in other suitable locations as would be readily apparent to a person skilled in the art.

The processing section 3 includes processing means for heating pressurised feedstock to supercritical temperatures. Typically, the feedstock will be heated to a temperature between 250° Celsius and 400° Celsius. However, it is envisaged that the system and process of the invention may also be used to process feedstock at temperatures outside this range.

It is envisaged also, that although in the preferred embodiment of the invention the feedstock is pressurised in the pressurising section, the feedstock may, alternatively or additionally, be pressurised or further pressurised in the processing section.

In the embodiment shown in FIG. 1, the processing means comprises a processing vessel 20 that includes a first stage 21 and a second stage 22, and a first end 30 and a second end 31 that substantially opposes the first end. An opening is positioned at or near the first end 30 of the pressure vessel and is connected to the outlet of the second valve 19.

The first stage of the pressure vessel comprises a first tube 21 having a first end 27 that connects with the opening to form an inlet 28 to the pressure vessel 20. The first tube 21 is positioned concentrically within a second tube 22 that forms the casing of the pressure vessel 20. A space 26 (preferably an annular space) is provided between the outer peripheral surfaces 21 of the first tube and the inner surfaces of the second tube 22. This space defines the second stage within the processing vessel 20 and leads to the outlet 24.

The first tube 21 is shorter than the processing vessel 20 and comprises a distal end 32 that terminates before the second end 31 of the processing vessel 20. A space is provided between the distal end 32 of the first tube 21 and the second end 31 of the processing vessel 20. This space forms a reaction zone or reaction chamber 23 where pressurised, high temperature feedstock reacts to form a raw product stream. The inlet 28, first stage 21, reaction zone 23, second stage 22, and outlet 24 form a fluid pathway along which the feedstock 7, and then the raw product stream 8, passes through the pressure vessel 20.

Each end 30, 31 of the processing vessel 20 is sealed, except where the inlet 28 enters the vessel 20 and where the outlet 24 exits the vessel. This arrangement allows the processing vessel to be used as a pressure vessel in which the same pressure is maintained within the vessel.

The inner and outer surfaces of both the first and second tubes 21, 22 are heat transfer surfaces.

In use, feedstock enters the first stage via the inlet 28. The feedstock moves through the fluid flowpath defined by the first stage and is heated before reaching the reaction zone 23, where the feedstock is further heated to a desired temperature by heating means 25 that causes the feedstock to react to form a raw product stream 8.

The heating means 25 is configured to heat the pressurised feedstock 7 in the reaction chamber 23 up to between 250° Celsius and 400° Celsius. The heating means 25 may be in the form of an element or other suitable heating means. The heating means 25 may be inserted directly into the reaction chamber 23 to heat the feedstock or it may be adapted to be located externally from the reaction chamber so as to heat the walls of the processing vessel 20 at or near the location of the reaction chamber 23.

The heating means 25 may heat the pressurised feedstock 7 in the reaction chamber 23 by radiation, convection, conduction, electromagnetic radiation, including microwave and ultrasonic radiation, or any combination of such heating methods or by similar heating methods.

The raw product stream and any unreacted feedstock then moves along the fluid flow path defined by the second stage 22 where the raw product stream is cooled to an ambient or near ambient temperature, preferably at or below 80° Celsius, before being discharged from the processing section 3 via the outlet 24.

In effect, the first and second tubes 21, 22 form a counter-flow heat exchanger, with the first tube 21 being made of a highly heat conductive material, such as a thin walled metal tube, to ensure a high heat transfer co-efficient. In addition, fins or other surface features that improve heat transfer may be incorporated onto or into the heat transfer surfaces of the processing vessel 20, tubes 21, 22 or reaction chamber 23.

The outlet 24 of the pressure vessel 20 is located on the periphery of the processing vessel 20 close to the inlet 28. However, it is envisaged that the outlet 24 could be located at other suitable locations on the processing vessel depending on the internal arrangement of the vessel, as would be apparent to a person skilled in the art.

In one form, the volume of the processing vessel 20 is at least six times that of the swept volume of the second pump 17. This volume difference enables the feedstock to be moved through the processing vessel in intermittent stages as the pump 17 is actuated. That is, one cycle of the pump 17 would cause a single charge of feedstock to move one sixth of the way through the processing vessel 20, thereby allowing for a longer residence time of the feedstock within the processing vessel 20 than if the same charge of feedstock was pushed into the processing vessel with the actuation of the pump 17 and was drawn out of the processing vessel with the next consecutive action of the pump. By allowing for a longer residence time, the feedstock is able to be heated to the desired temperature easily and is given sufficient time to undergo the desired reaction within the processing vessel.

As mentioned above, the first and second tubes 21, 22 of the processing vessel 20 are preferably concentric, with the first tube 21 being positioned inside the second tube 22 and defining an annular space 26 between. However, it is envisaged that the first and second stages of the processing vessel may be of different shapes and arrangements, as would be apparent to a person skilled in the art. For example, the processing vessel could comprise a housing having an inlet and an outlet and a counter-flow heat exchange system in between. Such arrangements are well known in the art and allow incoming feedstock to be heated by heating means and by outgoing feedstock that has already been heated. Similarly, the outgoing feedstock is cooled by the incoming feedstock and by being separated from or distanced from the heating means.

Alternatively, the processing vessel may comprise any other suitable arrangement by which the feedstock can be held under pressure whilst being heated and then cooled, as would be apparent to a person skilled in the art.

We turn now to the output section 4 of the system of the invention. The outlet 24 connects the processing vessel 20 to the output section 4 via a conduit. The discharged raw product stream moves along this conduit to the output section 4.

The output section 4 optionally includes a high pressure gas separator 40 for separating out gases from the raw product stream. In the embodiment in which a gas separator is used, the outlet 24 of the processing vessel 20 is connected with the inlet of the high pressure gas separator 40, which may be of a known type, so that the raw product stream moves from the processing vessel 20 to the gas separator 40 via a conduit. Any gases entrained, or formed in the processing vessel 20, and which remain within the feedstock, are able to exit the system by being purged from the gas separator through a purge valve 48 connected with the gas separator 40.

The output section also includes a third valve 41 that is connected with the outlet 24 of the processing vessel 20 or with an outlet 42 of the gas separator, if the gas separator is included within the system. The third valve 41 is also connected with a third pump 44.

The third pump 44 is a high pressure pump that acts as both a depressurising means and as a discharge pump. In particular, the third pump 44 comprises a pump housing in the form of a cylinder within which a floating third ram 45 is located. One side of the ram is in contact with the raw product stream as it enters the third pump. The other side of the ram is in contact with a pure fluid, such as water, which is the pressurised output of a separate conventional pumping system connected with the third pump 44. As the raw product stream 8 enters the cylinder via the open third valve 41, the ram presses against the pure fluid at the other end of the cylinder and the fluid is pushed out into a reservoir, not shown, via an open release valve at the pure fluid end of the cylinder.

The third valve 41 is controlled to open at the same time as the first valve 16 in the pressurising section 2. This allows a charge of product to leave the processing section 3 at the same time as a charge of feedstock enters the processing section 3, via the first valve 16, without significantly changing the pressure level in the processing section 3. The release valve acts to automatically maintain the pressure within the third pump 44 at about the same pressure as in the processing system 3, and as created by the pump action of the second pump 17 as the second pump transfers the charge of feedstock into the processing section 3. When the transfer of the new charge of feedstock 7 is complete and the transfer of the latest charge of product 8 is complete, both the second valve 19 and third valve 41 are closed. Further opening movement of the third ram 45 continues. This causes the capacity of the feedstock end of the cylinder to increase, thereby depressurising the feedstock. Preferably, the raw product stream is depressurised to ambient or near ambient levels.

Any gases that were dissolved in the raw product stream and that were not purged in the gas separation stage can then be ejected via a fourth valve 47, which is connected with the third pump 44 and which can also act to depressurise the raw product stream.

The third pump 44 is also connected with a fifth valve in the form of an outlet valve 46. This allows the depressurised raw product stream to be pumped, by actuation of the third pump 44, out through the outlet valve 46, which is opened to allow the raw product stream 8 to be discharged from the system.

Because the raw product stream is at an ambient or near ambient pressure, the outlet valve 46 is subject to less wear and is, therefore, more reliable than if the raw product stream was discharged through the outlet valve under high pressure.

Normally, the fourth valve 47 helps to reduce the pressure of the raw product stream in the third pump 44 after the third valve 41 has closed but before the outlet valve 46 has opened, so that rapid wear is avoided when the outlet valve 46 is opened.

It is envisaged that the system and process of the invention may be used with any suitable pumping systems, as would be appreciated by a person skilled in the art. However, the preferred form of pumping systems used in the present invention is unique in that the pumps are adapted to pressurise and depressurise the feedstock or raw product stream (as the case may be) by using indirect pressurising means and in that the pumps are able to pressurise the feedstock to high pressures and to depressurise the product to low pressures in an accurate way.

A particular advantage of using this form of pumping system is that the valves and sensitive component parts of the indirect pump are in contact with the fluid or water, which is clean, and are not in contact with the feedstock. This means that, unlike known system in the art, these component parts of the pump are less likely to become clogged and worn by the raw materials and chemicals within the feedstock that may be viscous, corrosive, dirty, or of a nature that is otherwise harmful to the component parts of the pumps and valves.

By pressurising a pure fluid, such as pure water, the indirect pump can pressurise the fluid and depressurise the fluid in an accurate way, rather than if the impure feedstock or raw product was pressurised directly. In effect, the pumps of the system of the invention can operate reliably to raise and lower the fluids to an accurate pressure because a clean fluid with known properties is being pressurised and so the level of pressurisation can take the known properties of the fluid into account. Therefore, the system and process of the invention can be more accurately controlled than known systems and methods, which pressurise the feedstock and raw product stream directly.

Although a pure fluid in the form of pure water is preferred within the pumps in the system of the invention, it is envisaged that other suitable fluids (even impure, but relatively clean fluids) may be used instead, as would be appreciated by a person skilled in the art.

An important feature of this invention is that when the raw product leaves the processing vessel 20 by means of the product outlet 24, the temperature of the raw product stream has been reduced to at or near ambient temperatures, preferably below 80° Celsius and more preferably below 50° Celsius. The third pump 44 and the valves 41, 46, and 47, in the outlet section 4 are, therefore, able to operate at near ambient temperatures with consequent advantages in: the performance of the components; low wear on the components; and prolonged equipment life. In particular, this results in the advantage that the equipment, valves, and pump components and materials can be chosen for their high sealing and pumping reliability and not simply because of their ability to withstand high temperatures (which is the limitation by which valves and component parts of pumps are chosen in prior art systems and processes).

In effect, the areas of the system of the invention where the pressures are raised in the second pump 17 with the associated valves, and the areas of the system where the pressures are brought down to ambient levels, are free to utilise highly efficient materials and components to provide good sealing performance, without risking the effectiveness of those materials and components by subjecting them to high temperatures. The valves and pumps can, therefore, be of any specification capable of withstanding high pressures and corrosive chemicals without needing to withstand high temperatures as well. Thus, the system of the invention avoids the need to use heat resistant valves in areas of high temperature.

Furthermore, the reaction zone 23, and the first and second stages 21, 22 of the processing means where the temperature is raised and lowered respectively, are maintained at constant pressure and can be made from suitable materials and components that can cope with these high temperatures without being required to use moveable seals, which would otherwise be required to change pressure and which are vulnerable to high temperatures.

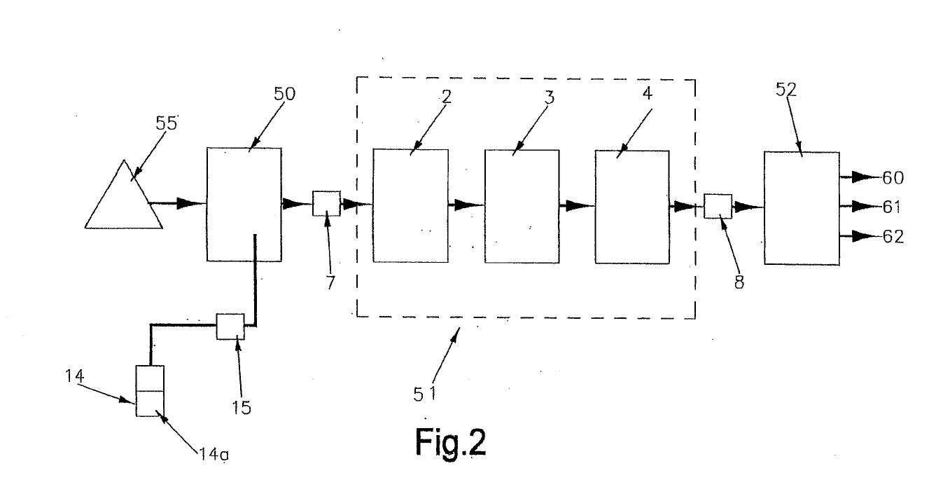

Referring now to the high pressure hydrothermal process of the invention, which is shown broken down into its main parts in FIG. 2, those main parts being: a preparation stage 50, a processing stage 51, and a separation stage 52.

The preparation stage 50 takes a raw material 55 to be processed and forms it into a pumpable feedstock or slurry 7.

The processing stage 51 pressurises and heats the feedstock or slurry to predetermined optimal temperatures and pressures to cause a reaction in the feedstock and to, thereby, produce a raw product stream as described in relation to the description of the invention above, then cools and depressurises the raw product in a controlled manner.

The separation stage 52 separates gas from the raw product stream 8.

Optionally, further separation can be conducted after the raw product stream is discharged from the outlet valve 46. In particular, the raw product stream may be separated to form one or more product sub-streams by solvent extraction, distillation, settling, membrane filtration, centrifuging, ion exchange, drying, evaporation, vacuum distillation/separation or any other suitable separation process or combination of processes as would be readily apparent to a person skilled in the art.

If the goal is to remove a contaminant from the feedstock 7, then there may be no need for the separation stage 52, depending on the nature of the contaminant.

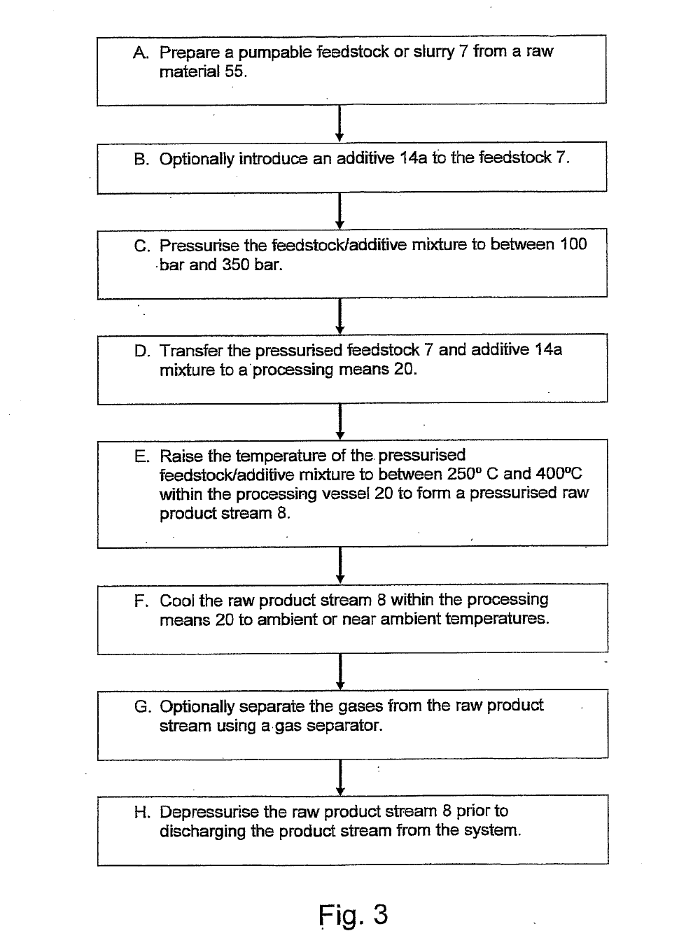

Referring to FIG. 3, a preferred process for using the high temperature hydrothermal system 1 is shown. The process is a continuous process (as opposed to a batch process) and includes the following steps, in order:
A. prepare a pumpable feedstock or slurry 7 from a raw material 55;
B. optionally introduce an additive 14a to the feedstock 7;
C. pressurise the feedstock or feedstock and additive mixture (as the case may be) to between 100 bar and 350 bar;
D. transfer the pressurised feedstock 7 and additive 14a mixture to a processing means, preferably in the form of a processing vessel 20;
E. raise the temperature of the pressurised feedstock 7 or feedstock and additive mixture to between 250° C. and 400° C. within the processing vessel 20 to form a pressurised raw product stream 8;
F. cool the raw product stream 8 to ambient or near ambient temperatures;
G. optionally separate the gases from the raw product stream using a gas separator; and
H. depressurise the raw product stream 8 prior to discharging the product stream from the system.

In step A, the raw material 55 is formed into a pumpable feedstock or slurry 7. The raw material 55 used in the system of the invention may be any organic or contaminated material such as de-lignitised timber, biomass, algae or drycleaning sludge.

Many of the raw materials will require some mechanical/thermal or chemical processing to break them down into a pumpable form of feedstock 7 or slurry, whilst other materials will require only the addition of water. The processes that could be used to break down the raw materials may include one or more of grinding, shredding, crushing, liquification, heating, chemical or thermal decomposition or similar.

For example, if drycleaning sludge is used as a raw material, the sludge is formed into a pumpable feedstock or slurry by the addition of between 30% to 50% water. If using cyanide waste as a raw material, no additional water is generally required. If using algae as a raw material, then between 1% and 30% water is left in the algae to produce a pumpable feedstock. Other materials, such as delignitised timber, may need to be mechanically broken down with some water required at times.

In a further embodiment, one or more additive(s) 14a is/are added to the raw material 55 to form the feedstock 7.

The pumpable feedstock 7 is then transferred to the feed tank 10.

In step B the first ram 12 draws a preset volume of feedstock 7 from the feed tank 10 through the non-return valve 13 into the first pump 11. At the same time and, optionally, the additive pump 15 pumps the required amount of one or more additives 14a from the additive tank 14 into the first pump 11 to form a feedstock and additive mixture.

The additive(s) 14a may be catalysts or reactants to help process the feedstock. The additive(s) used may include acids and bases. However, it is envisaged that oxidising and reducing agents may also be used. In another form, each additive may be chosen from the group of carbonates, hydroxides, bicarbonates and similar bases.

Although it has been found that certain processed materials do not benefit from any additive being used, these are generally limited to when the invention is used for decontamination purposes. Thus, the addition of one or more additives to the feedstock is an optional step in the process of the invention.

The first valve 16 is then opened and the feedstock 7 or feedstock and additive mixture (as the case may be) is transferred to the second pump 17 for the pressurisation stage.

In step C, the feedstock 7 or feedstock mixture inside the second pump 17 is pre-pressurised to about 9 bar by the first pump 11. The first valve 16 is then closed and the second ram 18 of the second pump further pressurises the feedstock/mixture to the required processing pressure of between 100 bar to 350 bar, preferably by using indirect pressurising means, as described above in relation to the system of the invention.

Once the feedstock/mixture inside the second pump 17 is at the desired pressure, step D is undertaken.

In step D, the second valve 19 is opened, and the pressurised feedstock 7 or feedstock mixture is pushed out of the second pump 17 and into the first tube 21 by the second ram 18. As the feedstock 7 or feedstock mixture passes along the inside of the first tube 21 towards the reaction chamber 23, it is preheated by heat transferred, from the raw product stream 8 moving along the second stage or annular space 26 of the processing vessel, via the heat transfer surfaces of the first tube 21.

In step E, the feedstock/mixture is pumped into the reaction chamber 23 and its temperature is adjusted to the desired processing temperature of between 250° Celsius and 400° Celsius by the heating means 25. The feedstock/mixture undergoes a reaction to form a raw product stream that then moves along the second stage of the processing vessel by moving along the annular space 26 provided between the walls of the first and second tubes 21, 22.

In step F, the high pressure raw product stream 8 is cooled, as it passes through the annular space 26, by the incoming feedstock 7 or feedstock and additive 14a mixture, as the case may be. Additional cooling may also occur by using a heat exchanger positioned between the processing vessel 20 and the separation column, if a gas separator 40 is used, or between the processing vessel 20 and the third valve 41, if no gas separator is used.

This cooled high pressure raw product stream 8 is preferably at an ambient or near ambient temperature that will not damage the third valve 41. For example, the raw product stream 8 is preferable cooled to a temperature of at or below 80° Celsius, and more preferably to about 50° Celsius (though this is dependent on the type of valve used).

The third valve 41 is then opened and the cooled raw product stream 8 exits the processing vessel 20 via the outlet 24 and is pumped to the third pump 44, or to the gas separator 40 if the system includes a gas separator.

In a further embodiment, optional step G includes the additional step of degassing the cooled high pressure raw product stream 8 in a gas separator 40 to remove any gas that is insoluble in that stream before the raw product is pumped to the third pump 44. The solubility of gases varies with temperature and pressure. Thus, by cooling the high pressure raw product stream 8, some gases may come out of the product. The gas formed is purged through a purge valve 48 or the like on the gas separator 40.

In step H, the third valve 41 is closed and the cooled high pressure raw product stream 8 within the third pump 44 is depressurised, by moving the third ram 45 and/or using a depressurising means 47. The depressurised product stream 8 can now be pumped out of the third pump 44 by opening the output valve 46 and using the third ram 45 to push the product stream 8 out and discharge the raw product from the system.

In one embodiment, the discharged raw product stream 8 may then pass through a product separator to be separated into one or more product sub-streams 60, 61, 62. This may be achieved by solvent extraction, distillation, settling, membrane filtration, centrifuging, ion exchange, drying, evaporation, vacuum distillation/separation or any other suitable separation process or combination of processes as would be readily apparent to a person skilled in the art.

In a preferred form, the system and process of the invention is used to produce a product sub-stream 60, 61, 62 in the form of a hydrocarbon oil rich stream, which can be used in place of crude oil or similar, for producing materials such as diesel, aviation fuel, lubricating oil, petrol, or similar products.

The system and process of the invention aim to match pressure on each side of the first, second, third, and fifth valves 16, 19, 41, and 46 to reduce wear on the valves and to thereby improve their reliability.

It should be noted that although described in a sequential manner, the valves 16, 19, 41, 46 are controlled and sequenced such that as one charge of feedstock 7 is drawn into the system 1 one charge of depressurised product stream 8 exits. Thus, the pumps are actuated simultaneously so as to produce a continuous process. Furthermore, the time delay between feeding new feedstock 7 into the system affects the residence time of feedstock within the processing vessel 20.

It has been found that the residence time influences the yield of oil and/or level of decontamination achieved. The preferred minimum time between charges is about 20 seconds. Thus, the minimum processing and/or residence time is the same as or greater than 20 seconds.

Although preferred forms of the invention are described as including a pump with a ram 12, 18, 45, it is envisaged that each of the pumps 11, 17, 44 can be any suitable pump or fluid transfer means known to those skilled in the art, such as a positive displacement pump, or a floating ram with one side connected to a high pressure pump of known type, or other mechanical means to accomplish the same role. For example, there may be more than one ram 12, 18, 45 and each of these may be used in sequence to generate a near continuous series of small charges passing through the system 1.

In a further embodiment, there is more than one first tube 21 connected to the second valve 19, making the processing vessel 20 somewhat like a shell-and-tube heat exchanger. In this form, the inlet 28 connects with a plurality of first tubes 21 within the processing vessel 20.

In a further embodiment, there can be more than one processing vessel 20 connected to the second valve 19.

It is also envisaged that the processing vessels may be of different lengths to suit the particular raw material used and to suit the desired residence time in the processing vessel and the desired outcome of the process. For example, in one embodiment, the invention could include six or more processing vessels, each being twice as long as the processing vessel of the embodiment shown in FIG. 1 and in parallel with each other. Each of these processing vessels may be equipped with an extra inlet valve in series with the individual inlets 28, so as to allow sequential charging of feedstock 7 with catalyst 14a.

The following list of examples gives some idea of the range of processing conditions and results achieved:

### EXAMPLE 1

Drycleaning sludge was the raw material processed in this example. This was the waste material from a drycleaning operation from which the majority of the perchloroethylene had been removed from the sludge prior to processing by the invention. The remaining concentration of perchloroethylene in the raw material was about 0.8%.

About 30% and 50% water was added to the raw material to form the feedstock. An additive of sodium hydroxide in water was then added to the feedstock. The feedstock and additive mixture was processed in the reaction chamber at about 280° Celsius and at a pressure of about 150 bar.

The time between charges was about 180 seconds and the processing vessel was 9 times the charge size.

Under these conditions, the resultant raw product stream had less than 30 ppm perchloroethylene remaining and it could be disposed of safely. Thus, the system and process of the invention helped to remove perchloroethylene that contaminated the drycleaning sludge.

### EXAMPLE 2

An inorganic cyanide waste was the raw material processed. No additional water or additive was needed.

The feedstock was processed at 280° Celsius+/−30° Celsius and at 150+/−50 bar with 180 seconds between charges.

The raw product included ammonia, which can be neutralised or recovered. The product stream was in need of further processing but no longer contained any significant amount of cyanide. Thus, the process of the invention helped to remove contaminating cyanide from the raw material

### EXAMPLE 3

In this example, algae was the raw material processed by the system and process of the invention. Between 1% and 30% water was left in the algae to form a pumpable feedstock.

An additive of 5% Na₂CO₃ was then added and the feedstock and additive mixture was processed at about 340° Celsius and at about 200 bar With 800 seconds between charges.

The raw product stream contained kerogen, which had a 37% yield of light crude oil and also contained waste that included Na₂CO₃. Thus, the system and process of the invention was used to produce material from which an alternative fuel similar to mineral crude oil could be produced.

### EXAMPLE 4

The raw material processed by the invention was coppiced de-lignitised willow chips, which were mechanically ground down prior to processing. Water was added to the raw material to form a pumpable feedstock and to this feedstock was added the additives of calcium carbonate and sodium carbonate.

The feedstock and additive mixture was processed at 320° Celsius and at 120 bar with a charge time of 1500 seconds.

The raw product stream yielded at least 2% mobile crude oil of a light low viscosity nature. Again, the system and process of the invention was used to produce a material from which an alternative fuel can be produced.

### EXAMPLE 5

The raw material processed by the invention was coppiced delignitised willow chips, which were mechanically ground down prior to processing. Water was added to the raw material to form a pumpable feedstock and to this feedstock was added the additives of calcium carbonate, sodium carbonate, and calcium hydroxide.

This feedstock and additive mixture was processed at 340° Celsius and at 150 bar.

The raw product stream provided an oil recovery rate of 6.4%.

### EXAMPLE 6

Waste water treatment plant biosolids were the raw material processed by the invention, using firstly no additive at 250° Celsius and, secondly, using an additive of sodium carbonate at 300° Celsius; both processes operating at a pressure of 150 bar with a charge time of 1500 seconds.

Without any additive, the yield was a very poor 0.9% oil recovery. But, when the process was used with the sodium carbonate additive, an oil recovery rate of 1.8% was achieved.

### EXAMPLE 7

In this example, the raw material processed was pure white cellulose fibre normally used for filter media. This was mixed with water and with additives of ground calcium carbonate powder, calcium hydroxide powder, and sodium carbonate powder. The slurry was then processed according to the invention.

The charge time was set at 1500 seconds, and the pressure was set at 200 bar.

During processing, the temperature of the mixture varied between 310° Celsius and 350° Celsius. Three runs were carried out.

It was discovered that a sludge was created, which was very thick and which collected as a deposit in the lower parts of the processing vessel without discharging from the outlet port. It is likely that the deposits were caused by an agglomeration of the minerals that were added and which had formed a hard lime cement. The deposits were finally removed by washing out the processing vessel with a toluene based solvent.

After settling out the solids, the resulting raw product stream comprised a black liquor that was then distilled at 120° Celsius to leave over 15% crude oil.

### EXAMPLE 8

A sample of kelp seaweed was obtained in a wet freshly harvested form and was used as the raw material to be processed by the invention. After draining out all free sea water, the kelp was reduced to a pumpable pulp in a Blackfriars mill. The analysis of dry mass at this stage was 18% dry matter, with the remainder being sea water.

This pulp feedstock was then mixed with an additive of calcium carbonate and processed at 320° Celsius at a pressure of 200 bar and with a charge time of 1500 seconds.

The raw product stream was a typical sludge that yielded a crude oil after extraction with a solvent.

### EXAMPLE 9

In a new major development, a second much larger hydrothermal reactor system according to the invention was constructed based on the same technology. This mark 2 plant utilised identical pressure raising and pressure reducing technology and components but used six reaction vessels arranged in parallel, each processing vessel being identical to the processing vessel/pressure vessel 20 shown in FIG. 1.

Initial testing with an algae feedstock has yielded a raw product stream with an average of 18% oil content using algae as a dry matter feedstock. The operating conditions used a temperature of approximately 320° Celsius, a pressure of 150 bar, and a 700 second cycle time. An additive of about 5% sodium carbonate was mixed with the algae feedstock before processing.

Overall the best results so far for oil production have been to add sodium carbonate or calcium carbonate to the feedstock to form a feedstock and additive mixture that contains at least 5% additive.

Variations and modifications to the preferred embodiments of the invention described herein will be apparent to those skilled in the art. It is intended that such variations and modifications may be made without departing from the scope of the invention and without diminishing its attendant advantages.

What we claim is:

1. A system 1 for processing a raw material in the form of a pumpable feedstock, the system comprising:
a) a pressurising section 2 comprising pressurising means 17 for pressurising the feedstock 7 to a pressure in the range of 100 to 400 bar;
b) a processing section 3 comprising processing means 20 for heating the feedstock 7 to a temperature in the range of 250° to 400° C. by heating means 25 to form a raw product stream 8 and in which the processing means is adapted to allow the raw product stream 8 to be cooled to an ambient or near ambient temperature before exiting the processing section 3; and
c) an output section 4 in which the raw product stream 8 is depressurised by depressurising means 44.

2. The system of claim 1, wherein the pressurising section 2 comprises a first pump and wherein the pressurising means comprises a second pump 17, the first pump 11 being configured to draw feedstock 7 from a feed tank 10 and provide an initial low pressurisation and the second pump 17 being configured to pressurise the feedstock 7.

3. The system of claim 2, wherein the pressurising section 2 further comprises an additive pump 15 for adding one or more additives 14a to the feedstock 7 to form a feedstock and additive mixture before the mixture is pressurised by the second pump 17.

4. The system of any one of claims 1 to 3, wherein the processing means comprises at least one processing vessel 20 comprising an inlet 28 to a first stage 21 connected with a reaction zone 23 that is heated by the heating means 25, the reaction zone 23 being connected to a second stage 22 leading to an outlet 24 through which reacted feedstock, in the form of a raw product stream 8, is able to exit the pressure vessel 20, and wherein the inlet 28, first stage 21, reaction zone 23, second stage 22, and outlet 24 form a fluid pathway along which the feedstock 7, and then the raw product stream 8, can pass through the pressure vessel 20.

5. The system of claim 4, wherein the processing vessel comprises a pressure vessel 20 comprising a first end 30 and a second end 31, wherein an inlet 28 is positioned at or near the first end 30 of the pressure vessel 20, the inlet 28 being connected to a first tube 21 positioned concentrically within a second tube 22, wherein the second-tube 22 forms the casing of the pressure vessel 20; and wherein the first tube 21 comprises a distal end 32 that terminates before the second end 31 of the pressure vessel 20 to form a reaction zone 23 between the distal end 32 of the first tube 21 and the second end 31 of the pressure vessel 20.

6. The system of any preceding claim, wherein the processing means 20 comprises a counter-flow heat exchanger.

7. The system of any preceding claim, wherein the output section 4 comprises a high pressure gas separator 40 for separating out gases from the raw product stream 8, and further comprises a purge valve 48 through which the gases can exit the system 1.

8. The system of claim 2, wherein the second pump 17 is adapted to pressurise the feedstock 7 using indirect pressurisation, the second pump 17 comprising a housing in which a floating ram 18 is located, wherein one side of the ram 18 is adapted to be in contact with a fluid output of a separate pumping system, and the other side of the ram 18 is adapted to be in contact with the feedstock 7 within the housing, such that pressurising the fluid within the housing causes the ram 18 to press against the feedstock 7 to pressurise the feedstock

9. The system of any preceding claim, wherein the depressurising means comprises a third pump 44 adapted to depressurise the product stream 8 using indirect depressurisation, the third pump 44 comprising a housing in which a floating ram 45 is located, wherein one side of the ram 45 is adapted to be in contact with a fluid output of a separate pumping system, and the other side of the ram 45 is adapted to be in contact with the raw product stream 8, and wherein the housing includes a release valve through which the fluid within the housing can exit the housing to allow the raw product stream to be depressurised.

10. A continuous process for treating a raw material, the process comprising the following steps, in order:
a) prepare a pumpable feedstock or slurry 7 from a raw material 55;
b) pressurise a charge of the feedstock 7 to between 100 bar and 350 bar;
c) transfer the pressurised feedstock 7 to a processing means 20;
d) raise the temperature of the pressurised feedstock 7 to between 250° C. and 400° C. within the processing means 20 to form a pressurised raw product stream 8;
e) cool the raw product stream 8 within the processing means 20 to ambient or near ambient temperatures; and
f) depressurise the raw product stream 8 before discharging the raw product stream from the system.

11. The process of claim 10, wherein the raw product stream 8 is cooled in the processing means 20 to a temperature below 80° C.

12. The process of claim 10, wherein the raw product stream 8 is cooled in the processing means 20 to a temperature below 50° C.

13. The process of any one of claims 10 to 12, wherein one or more additives 14a are introduced to the pumpable feedstock 7 before the feedstock and additive mixture is pressurised.

14. The process of claim 13, wherein each additive is selected from the group consisting of: acids, bases, oxidising agents, and reducing agents.

15. The process of claim 13, wherein each additive is selected from the group consisting of: carbonates, hydroxides, bicarbonates, and similar bases.

16. The process of claim 13, wherein an additive 14a of sodium carbonate or calcium carbonate is added to the feedstock 7 to form a feedstock and additive mixture containing at least 5% additive.

17. The process of any one of claims 10 to 16, wherein the feedstock 7 is pressurised by indirect pressurisation.

18. The process of any one of claims 11 to 17, wherein the raw product stream 8 that exits the processing means 20 is then passed through a gas separator 40 in which gas is purged from the raw product stream.

19. The process of any one of claims 10 to 18, wherein the processing time between charges of feedstock 7 is a minimum of 20 seconds.

20. The process of claim 10, wherein the feedstock comprises algae to which is added an additive of calcium carbonate to form a feedstock and additive mixture, which is then pressurised to 150 bar and heated to a temperature of approximately 320° C.
